(19) United States
(12) Patent Application Publication
(10) Pub. No.: US 2022/0170944 A1
(43) Pub. Date: Jun. 2, 2022

(54) MATERIALS AND METHODS FOR IDENTIFYING AND TREATING EOSINOPHILIC DISORDERS

(71) Applicant: The Regents of the University of California, Oakland, CA (US)

(72) Inventors: Ranjan Dohil, San Diego, CA (US); Seema S. Aceves, Solana Beach, CA (US)

(21) Appl. No.: 17/310,265

(22) PCT Filed: Jan. 31, 2020
(86) PCT No.: PCT/US2020/016287

§ 371 Date: Jul. 27, 2021

Related U.S. Application Data
(60) Provisional application No. 62/799,716, filed on Jan. 31, 2019.

(51) Int. Cl. G01N 33/68; G01N 1/30; C12Q 1/37

(52) U.S. Cl. CPC G01N 33/6893; G01N 1/30; G01N 2333/8132; G01N 2800/52; G01N 2800/24; C12Q 1/37

(57) ABSTRACT

The disclosure relates to diagnostic and biomarker panels for determining an eosinophilic disease or disorder. More particularly, the disclosure provides methods and diagnostics for determining eosinophilic esophagitis.

### CROSS REFERENCE TO RELATED APPLICATIONS

This application is a U.S. National Phase Application filed under 35 U.S.C. § 371 and claim priority to International Application No. PCT/US2020/016287, filed Jan. 31, 2020, which application claims priority under 35 U.S.C. § 119 to U.S. Provisional Application Ser. No. 62/799,716, filed Jan. 31, 2019, the disclosures of which are incorporated herein by reference.

### STATEMENT REGARDING FEDERALLY SUPPORTED RESEARCH

The invention was funded by grant numbers AI092135 and AI135034 awarded by the National Institutes of Health. The government has certain rights in the invention.

### FIELD OF THE INVENTION

The invention relates to the methods and compositions for determining the presence of eosinophilic disorders and methods of treating eosinophilic disorders.

### BACKGROUND

Esophageal inflammation disorders are gaining increased recognition in both adults and children. One example is eosinophilic esophagitis (EE or EoE), which is an emerging and fast-growing disorder characterized by high levels of eosinophils in the esophagus, as well as basal zone hyperplasia and esophageal remodeling that includes fibrosis and smooth muscle dysfunction leading to complications of strictures and food impactions. EoE is thought to be provoked, in at least a subset of patients, by food allergies or airborne allergen exposure. EoE diagnosis is often associated with other hypersensitivity disorders, including asthma, rhinitis, and other food and aeroallergen inhalant sensitivities. Diagnosis is often made, e.g., in young children and depends on the finding of 15 or more eosinophils per high power field (eos/hpf) within esophageal mucosal biopsies.

In parallel with other atopic disorders, the incidence of EoE appears to be increasing. The disorder may present with reflux-like symptoms, pain and dysphagia, clinical symptoms similar to the presentation of gastroesophageal reflux disease (“GERD”). Symptoms of EoE may include, for example, one or more of the following: abdominal pain, chest pain, choking, difficulty swallowing, failure to thrive, nausea, reflux, vomiting, and weight loss. In one series, 15% of EoE patients had concurrent developmental delay.

Although EoE is becoming more frequently diagnosed throughout developing countries many aspects of the disease remain unclear including its etiology, natural history and optimal therapy. Symptoms of EoE often mimic those of GERD and include vomiting, dysphagia, pain and food impaction. However, treatment of EoE and GERD differ and it is important to distinguish between them, particularly as untreated EoE may be associated with esophageal narrowing in 10-30% of cases and >70% of adults progress to strictures if untreated. The common occurrence regarding misdiagnosis of EoE for GERD often results in delayed treatment for patients with EoE.

### SUMMARY

The disclosure provides a method of determining an eosinophilic disease or disorder status in a subject, comprising measuring the levels of a biomarker panel in a sample from the subject that comprises at least one polypeptide, marker or gene selected from the group consisting of COMP-1, tryptase, PAI-1 and any combination thereof; analyzing the result to determine a level of expression of the at least one polypeptide or gene; and determining the eosinophilic disease or disorder status of the subject based upon the level of the expression or marker. In one embodiment, the status comprises a diagnosis of eosinophilic esophagitis. In another embodiment, the at least one marker or gene comprises mRNA. In yet another embodiment, the at least one marker or gene comprises protein. In any of the foregoing embodiments, the subject is a human patient. In another or further embodiment, the sample is obtained from the group consisting of a tissue, an exudate, saliva, serum, plasma, blood, oral, urine, stool, and a buccal sample. In a further embodiment, the sample is a tissue sample. In still a further embodiment, the tissue sample is a esophageal tissue sample. In another embodiment, the determining step comprises analyzing a subset of the markers or genes using at least one algorithm. In still another embodiment, the status comprises distinguishing eosinophilic esophagitis from a normal condition in the subject. In a further embodiment, the distinguishing comprises determining the percent of PAI-1, COMP1 and/or tryptase in the sample. In yet a further embodiment, the method comprises determining if the amount of PAI-1 is greater than a threshold level. In a further embodiment, the threshold level is greater than 14% as determined by PAI-1 staining in a sample. In still a further embodiment, the method includes identifying a subject having a sample with greater than 14% PAI-1 staining as having active eosinophilic disease and/or high probability of fibrosis. In still a further embodiment, the method includes identifying the subject with high PAI-1 as a candidate subject for treatment with an aggressive therapeutic regimen. In some embodiments, the therapeutic regiment is treatment with a combination of steroids and TZD(s). In yet another embodiment, the status comprises distinguishing eosinophilic esophagitis from at least one other eosinophilic disorder in the subject. In yet another embodiment, the method further comprises developing or modifying a therapy for the subject based upon the results of the diagnostic panel analysis. In another embodiment, the sample is an archival sample. In a further embodiment, the archival sample is a formalin-fixed, paraffin-embedded (FFPE) sample. In still another embodiment, the method further comprises determining and/or monitoring exposure to one or more therapeutic compounds in the subject based upon the level of expression.

The disclosure also provides an EoE molecular diagnostic panel comprising at least one marker detection agent that detects a marker selected from COMP-1, tryptase and PAI-1. In one embodiment, the detection agent is an antibody. In another embodiment, the detection agent comprises an oligonucleotide primer and/or probe.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an embodiment of the invention. Also, aspects described as a genus or selecting a member of a genus, should be understood to embrace combinations of two or more members of the genus. Variations of the invention defined by such amended paragraphs also are intended as aspects of the invention.

### BRIEF DESCRIPTION OF THE FIGURES

WM FIG. 1 provides histological stains of PAI-1, COMP, and tryptase in the epithelium of normal (left) and EoE (right) tissue (red stain indicates positive cells).

FIG. 2 provides correlation coefficients for PAI-1, COMP, and tryptase with fibrosis scores.

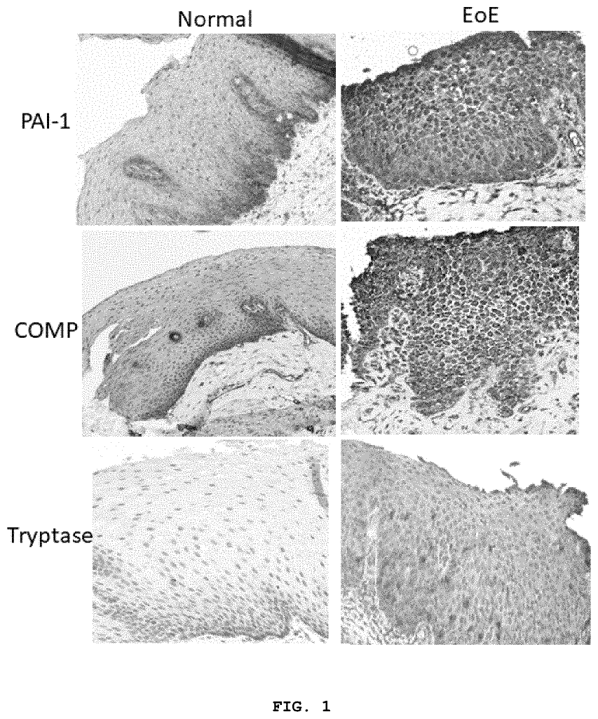

FIG. 3 is a collagen 6 stain (red) in the lamina propria of EoE and normal biopsies.

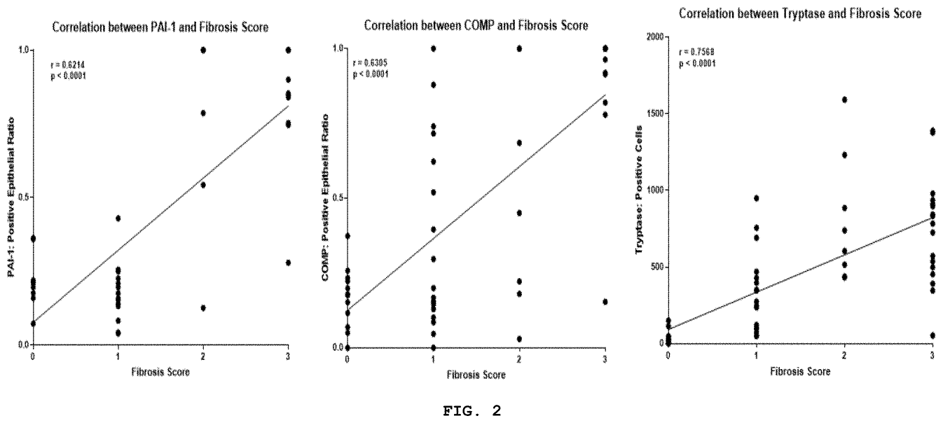

FIG. 4 shows PAI-1 Staining Distinguishes Normal and Abnormal Compliance. EoE patients with normal and abnormal compliance grouped by PAI-1 staining (p=0.04). The vertical axis shows the percent of epithelium that stained positively.

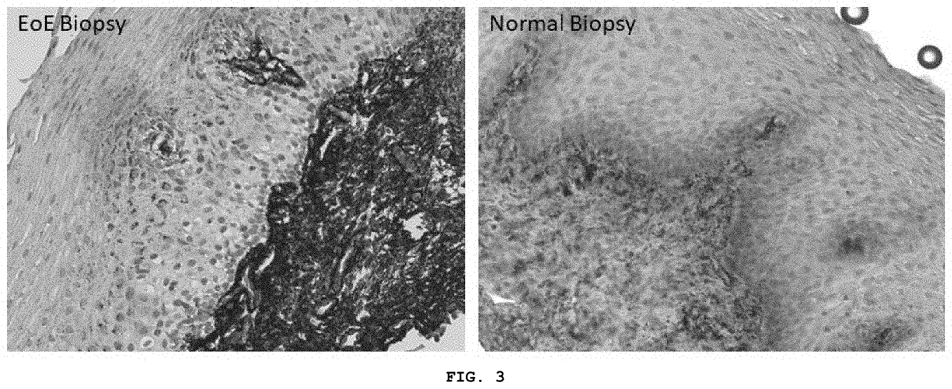

FIG. 5 shows PAI-1 Staining Distinguishes Normal and Abnormal Compliance. EoE and control patients with normal and abnormal compliance grouped by PAI-1 staining (p=0.04). The vertical axis shows the percent of epithelium that stained positively.

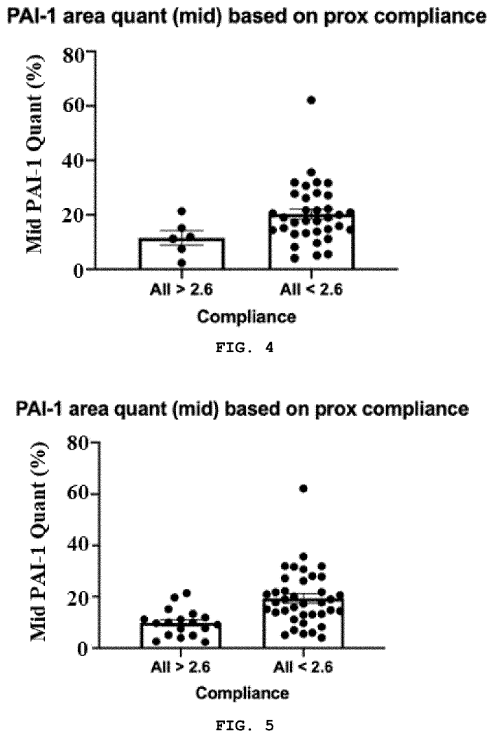

### DETAILED DESCRIPTION

As used herein and in the appended claims, the singular forms “a,” “an,” and “the” include plural referents unless the context clearly dictates otherwise. Thus, for example, reference to “a protein” includes a plurality of such proteins and reference to “a patient” includes reference to one or more patients and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and products, the exemplary methods, devices and materials are described herein.

The documents discussed above and throughout the text are provided solely for their disclosure prior to the filing date of the present application. Nothing herein is to be construed as an admission that the inventors are not entitled to antedate such disclosure by virtue of prior disclosure. Each document is incorporated by reference in its entirety with particular attention to the disclosure for which it is cited.

The following references provide one of skill with a general definition of many of the terms used in this disclosure: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

As used herein, the term “assessing” includes any form of measurement, and includes determining if an element is present or not. The terms “determining,” “measuring,” “evaluating,” “assessing” and “assaying” can be used interchangeably and can include quantitative and/or qualitative determinations.

The term “Cartilage Oligomeric Matrix Protein-1” or “COMP-1” refers to a noncollagenous extracellular matrix (ECM) protein having identical glycoprotein subunit monomers, each monomer with EGF-like and calcium-binding (thrombospondin-like) domains. Structurally, “Cartilage Oligomeric Matrix Protein-1” or “COMP-1” refers to nucleic acids and polypeptide polymorphic variants, alleles, mutants, and interspecies homologs that: (1) have an amino acid sequence that has greater than about 90% amino acid sequence identity, for example, 91%, 92%, 93%, 94%, 95%, 96%, 97%, 98% or 99% or greater amino acid sequence identity, preferably over a region of at least about 25, 50, 100, 200, 400, or more amino acids, or over the full-length, to an amino acid sequence encoded by a COMP nucleic acid (see, e.g., GenBank Accession No. NM000095.2); (2) bind to antibodies, e.g., polyclonal antibodies, raised against an immunogen comprising an amino acid sequence of a COMP polypeptide (e.g., GenBank Accession No. NP000086.2); or an amino acid sequence encoded by a COMP nucleic acid (e.g., COMP polynucleotides described herein), and conservatively modified variants thereof; (3) specifically hybridize under stringent hybridization conditions to an anti-sense strand corresponding to a nucleic acid sequence encoding a COMP protein, and conservatively modified variants thereof; (4) have a nucleic acid sequence that has greater than about 90%, preferably greater than about 91%, 92%, 93%, 94%, 95%, 96%, 97%, 98%, 99%, or higher nucleotide sequence identity, preferably over a region of at least about 25, 50, 100, 200, 500, 1000, 2000 or more nucleotides, or over the full-length, to a COMP nucleic acid (e.g., COMP polynucleotides, as described herein, and COMP polynucleotides that encode COMP polypeptides, as described herein).

“Diagnostic” means identifying or assessing the presence, extent and/or nature of a pathologic condition. Diagnostic methods differ in their specificity and selectivity. While a particular diagnostic method may not provide a definitive diagnosis of a condition, it suffices if the method provides a positive indication that aids in diagnosis.

As used herein, the term “eosinophilic disease and disorders” include a set of disease and disorders that comprise abnormal eosinophil concentrations and activity. It has been shown that eosinophils have been found to be increased or pathologically present in various conditions, including skin and subcutaneous disorders, pulmonary conditions, gastrointestinal diseases, neurologic disorders, cardiac conditions and renal disease. Exemplary skin and subcutaneous disorders in which eosinophils have been found to be pathologically present include, but are not limited to, atopic dermatitis (eczema), bullous pemphigoid, pemphigus vulgaris, dermatitis herpetiformis, drug-induced lesions, urticaria, eosinophilic panniculitis, angioedema with eosinophilia, Kimura's disease, Shulman's syndrome, Well's syndrome, eosinophilic ulcer of the oral mucosa, eosinophilic pustular folliculitis, and recurrent cutaneous necrotizing eosinophilic vasculitis. (See, e.g., Simon et al. J Allergy Clin Immunol. 2010 July; 126(1): 3-13). Exemplary pulmonary conditions associated with pathologically present eosinophils include, but are not limited to, drug/toxin-induced eosinophilic lung disease, Loeffler's syndrome, allergic brochopulmonary aspergillosis, eosinophilic pneumonia, Churg-Strauss syndrome, eosinophilic granuloma and pleural eosinophilia. Exemplary gastrointestinal diseases associated with pathologically present eosinophils include, but are not limited to, gastroesophageal reflux, parasitic infections, fungal infections, Helicobacter pylori infections, inflammatory bowel disease (ulcerative colitis and Crohn's disease), food allergic disorders, protein-induced enteropathy and protein-induced enterocolitis, allergic colitis, celiac disease, pemphigus vegetans (MR) and primary eosinophilic esophagitis, gastroenteritis, and colitis. Exemplary neurologic disorders associated with pathologically present eosinophils include, but are not limited to, organizing chronic subdural hematoma membranes, central nervous system infections, ventriculoperitoneal shunts, and drug-induced adverse reactions. Exemplary cardiac conditions associated with pathologically present eosinophils include, but are not limited to, Secondary to systemic disorders such as the hypereosinophilic syndrome or the Churg-Strauss syndrome, heart damage has been reported. It is also known that certain congenital heart conditions (such as septal defects, aortic stenosis) are associated with increased levels of eosinophils in the blood. Exemplary renal diseases associated with pathologically present eosinophils include, but are not limited to, interstitial nephritis and eosinophilic cystitis.

As used herein, the term “expression levels” refers, for example, to a determined level of biomarker expression. The term “pattern of expression levels” refers to a determined level of biomarker expression compared either to a reference (e.g. a housekeeping gene or inversely regulated genes, or other reference biomarker) or to a computed average expression value (e.g. in DNA-chip analyses). A pattern is not limited to the comparison of two biomarkers but is more related to multiple comparisons of biomarkers to reference biomarkers or samples. A certain “pattern of expression levels” can also result and be determined by comparison and measurement of several biomarkers as disclosed herein and display the relative abundance of these transcripts to each other.

As used herein, the term “marker” or “biomarker” refers to a biological molecule, such as, for example, a nucleic acid, peptide, protein, hormone, and the like, whose presence or concentration can be detected and optionally correlated with a condition, such as a disease state. It can also be used to refer to a differentially expressed gene whose expression pattern can be utilized as part of a predictive, prognostic or diagnostic process in healthy conditions or a disease state, or which, alternatively, can be used in methods for identifying a useful treatment or prevention therapy.

Plasminogen activator inhibitor (PAI)-1 is a serine proteinase inhibitor, which is the primary physiological inhibitor of plasminogen activation in vivo, and thus is a primary regulator of the fibrinolytic system. PAI-1 has a functional role in wound healing, atherosclerosis, metabolic disturbances (such as obesity and insulin resistance), tumor angiogenesis, chronic stress, bone remodeling, asthma, rheumatoid arthritis, fibrosis, glomerulonephritis and sepsis.

The term “PAI-1” includes any PAI-1 gene, cDNA, mRNA, or protein from any organism and that is a PAI-1 that can reduce or inhibit plasminogen activator. For example, GenBank Accession Nos: X04744 and CAA28444 disclose human PAI-1 nucleic acid and protein sequences, respectively and GenBank Accession Nos: M33960 and AAA39887 disclose mouse PAI-1 nucleic acid and protein sequences, respectively.

In one example, a PAI-1 sequence includes a full-length wild-type (or native) sequence, as well as PAI-1 allelic variants, variants, fragments, homologs or fusion sequences that retain the ability to reduce or inhibit plasminogen activator. In certain examples, PAI-1 has at least 80% sequence identity, for example at least 85%, at least 90%, at least 95%, or at least 98% sequence identity to a native PAI-1. In other examples, PAI-1 has a sequence that hybridizes under very high stringency conditions to a sequence set forth in GenBank Accession No. X04744 or M33960, and retains PAI-1 activity. In yet other examples, a PAI-1 protein has a sequence that can bind to a PAI-1 antibody.

As used herein, a “reference pattern of expression levels” refers to any pattern of expression levels that can be used for the comparison to another pattern of expression levels. In some embodiments of the disclosure, a reference pattern of expression levels is, for example, an average pattern of expression levels observed in a group of healthy or diseased individuals, serving as a reference group.

As used herein, the term “sample” encompasses a sample obtained from a subject or patient. The sample can be of any biological tissue or fluid. Such samples include, but are not limited to, sputum, saliva, buccal sample, oral sample, blood, serum, mucus, plasma, urine, blood cells (e.g., white cells), circulating cells (e.g. stem cells or endothelial cells in the blood), tissue, core or fine needle biopsy samples, cell-containing body fluids, free floating nucleic acids, urine, stool, peritoneal fluid, and pleural fluid, liquor cerebrospinalis, tear fluid, or cells therefrom. Samples can also include sections of tissues such as frozen or fixed sections taken for histological purposes or microdissected cells or extracellular parts thereof. A sample to be analyzed can be tissue material from a tissue biopsy obtained by aspiration or punch, excision or by any other surgical method leading to biopsy or resected cellular material. In some embodiments, the sample can be a saline swish, a buccal scrape, a buccal swab, and the like. In one embodiment, the sample is obtained by a buccal swab or esophageal swab. Proteins and/or nucleic acids can be isolated from the swab and the level of, e.g., PAI-1, COMP-1 and/or tryptase expression can be determined using RT-PCR, antibodies or nucleic acid or protein chips depending upon the type of biological material isolated and desired (e.g., protein or nucleic acids).

As used herein, the term “subject” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender. In various embodiments the subject is human. In some embodiments, the subject is a human child having an age of 16 years or less. Administration of a therapeutic described herein to a child having an age of 16, 15, 14, 13, 12, 11, 10, 9, 8, 7, 6, 5, 4, 3, 2, 1 or younger is specifically contemplated. In some embodiments, the subject is 12-17 years old, 12-16 years old, or 12-15 years old, 2-9 years old, or 10-18 years old. In some embodiments, the subject is an adult human. In other embodiments, the subject is 17 or more years old. In some embodiments, the subject is an infant human of about 1-12 months of age. In another embodiment, the subject is a toddler of about 1-3 years of age. In yet another embodiment, the subject is a human child of primary school age (e.g., about 4-11 years of age). In still another embodiment, the subject is an adolescent of about 12-18 years of age.

As mentioned above Eosinophilic Esophagitis (EoE) is now a relatively common disorder which, up to about 20 yrs years ago, was rarely reported. Now, between 1 in 500-1000 people are believed to suffer with this disorder. Diagnosis and monitoring of this disease is challenging and invariably requires invasive endoscopic testing with biopsies. The latter, however, may not be adequate to identify the development of esophageal fibrosis especially at an early stage in its development. Esophageal fibrosis may ultimately lead to esophageal stricture formation, significant symptoms and impact to quality of life.

Esophageal diseases including Eosinophilic Esophagitis (EoE) are associated with fibrosis and tissue remodeling that leads to eventual organ dysfunction. The tools required to gauge fibrosis or esophageal function are invasive and not all patients or tissue can be assessed for remodeling events such as fibrosis due to the paucity of deep tissue. Evaluating the presence of esophageal fibrosis is very difficult as this process usually involves deeper inaccessible tissues. A surrogate marker for fibrosis, particularly for early potentially reversible fibrosis, would be valuable in clinical practice in many different organs.

EoE progresses almost uniformly to esophageal narrowing (strictures) through the process of tissue remodeling which consists of fibrosis as well as increased vascularity and smooth muscle. Physiologic remodeling is reversible while pathological remodeling is often not. The fibrostenotic esophagus is resistant to standard therapies. However, the current ability to distinguish the progression of remodeling is challenging. Paucity of deep tissue in the biopsies limits gauging disease severity. A second manner to determine EoE complication and severity is through the endoscopic functional lumen imaging probe (endoFLIP) which measures the cross sectional area, distensibility and compliance of the esophagus. However, this is considered a research tool and not available to the majority of patients. Since esophageal fibrosis is an important marker of rigidity and rigidity reflects the progressive pathway to esophageal stricture formation, there is a significant clinical need to measure EoE complications. Further, since >70% of adult EoE patients are diagnosed when a stricture is already in place, the ability to treat and gauge fibrotic response is needed. Early detection of fibrosis with appropriate therapy may alter the natural course of the fibrotic process and prevent the development of severe scarring/stricture formation or even malignancy.

There are presently a number of therapies that are available for the treatment of EoE. These include dietary manipulation and topical steroids such as oral viscous budesonide (OVB). Other potential therapies which would constitute a more “aggressive” approach and therefore perhaps more likely to reduce esophageal fibrosis and stricture formation would include therapy with OVB/rosiglitazone, systemic corticosteroids, and biologics such as reslizumab, dupilumab and anti-IL13. The methods provided herein can help to define which patients would require which therapy, at an early enough stage to actually reverse the fibrosis and stricture development. The panel could then be used to monitor response to therapy.

The disclosure provides methods and composition useful to identify eosinophilic-related diseases and disorder. The disclosure exemplifies the methods and compositions as they related to the esophagus, but, could be useful in other organs. In addition, the methods of determining eosinophilic-related diseases and described herein can be combined with selective treatment modalities as further discussed herein. For example, wherein an eosinophilic disease or disorder is “active” (e.g., active EoE), the subject can be treated with a more aggressive therapy to reduce the advancement of the disease to fibrosis. For example, if active EoE is identified the sbuect can be treated using corticosteroids and Thiazolidinediones in combination thereby targeting both the inflammatory response and the fibrotic response.

In some embodiments, corticosteroids are selected from, by way of non-limiting example, aclometasone, amcinomide, beclometasone, betamethasone, budesonide, ciclesonide, clobetasol, clobetasone, clocortolone, cloprednol, cortivazol, deflazacort, deoxycorticosterone, desonide desoximetasone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluticasone, fluticasone propionate, fuprednidene, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, remexolone, tixocortol, triamcinolone and ulobetasol, and combinations, pharmaceutically acceptable salts and esters thereof. In certain embodiments, the corticosteroids used in the disclosure comprise topical steroids including, for example, budesonide. Budesonide is a synthetic corticosteroid which has been effective for the treatment of inflammatory diseases of the gastrointestinal tract such as EoE, Crohn's disease and ulcerative colitis. The chemical name of budesonide is 16,17-(butylidenebis(oxy))-11,21-dihydroxy-, (11-β,16-α)-pregna-1,4-diene-3,20-dione, and its chemical structure is:

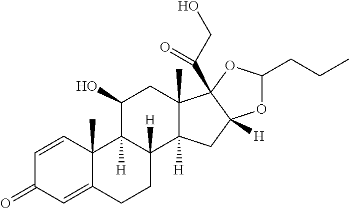

Corticosteroids (e.g., budesonide) when administered in oral form, in a formulation with increased coating (e.g., viscosity and/or mucoadhesive) characteristic, has been shown to be effective at reducing the inflammation of the esophagus.

Thiazolidinediones (TZDs), are a class of compounds which work by enhancing insulin action and promoting glucose utilization in peripheral tissue. TZDs include compounds known in the art as “TZD derivatives.” TZDs have no effect on insulin secretion. They apparently work by enhancing insulin action and thus promoting glucose utilization in peripheral tissues, possibly by stimulating non-oxidative glucose metabolism in muscle, and suppressing gluconeogenesis in the liver. The chemical compounds that comprise the Thiazolidinedione (TZD) class of compounds is exceptionally large. See, for example, Bowen, et al. Metabolism 40:1025 (1991); Chang, et al Diabetes 32:630 (1983); Colca, et al. Metabolism 37:276 (1988); Diani, et al. Diabetologia 27:225 (1984); Fujita, et al. Diabetes 32:804 (1983); Fujiwara, et al. Diabetes 37:1549 (1988). Exemplary of the family of thiazolidinediones are troglitazone, ciglitazone, pioglitazone (see U.S. Pat. Nos. 4,687,777 and 4,287,200), englitazone, CS-045[(±)-5[4-(6-hydroxy-2,5,7,8-tetramethylchroman-2-YL-methoxy) benzyl]-2,4-thiazolidinedione], TZD 300512, and BRL 49653. The thiazolidinediones (TZD), such as the FDA-approved anti-diabetic drugs rosiglitazone and pioglitazone, function as agonists for the ligand-activated nuclear receptor peroxisome proliferator-activated receptor-γ (PPAR-γ). PPAR-γ is a key regulator of lipid and glucose metabolism expressed on multiple cell types that can have anti-fibrotic, pro-adipogenic, and anti-inflammatory effects in structural and immune cells. Rosiglitazone is effective in the treatment of mild to moderately active ulcerative colitis. Pioglitazone, a more clinically favorable TZD, improves liver fibrosis in adults with nonalcoholic steatohepatitis (NASH), achieving resolution of NASH in up to 51% of patients. In addition, pioglitazone was reported to be well tolerated and had no major drug-related adverse events when compared to placebo. PPAR gamma agonists such as rosiglitazone and pioglitazone, metformin, pentoxyfylline, vitamin E, selenium, omega-3 fatty acids and betaine are of particular interest.

Given the natural trajectory of EoE towards pathologic remodeling with fibrosis, it was hypothesized that TZDs might decrease fibrotic gene and protein expression in esophageal fibroblasts. The disclosure demonstrates the differential expression of PPAR-γ in EoE versus normal esophagi and the ability of the TZDs to reduce fibrotic and myofibroblast gene and protein expression in EoE-derived esophageal fibroblasts, setting forth their use as therapeutic agents to treat the fibrotic diseases including fibrosis of the esophagus in EoE.

The disclosure uses a panel of markers to stain the epithelium from superficial mucosal biopsies (e.g., esophagus mucosal biopsies). The panel of markers includes, but, is not limited to plasminogen activator inhibitor-1 (PAI; serine proteinase inhibitor E1/SERPINE1), cartilage oligomeric matrix protein-1 (COMP-1/thrombospondin-5), and tryptase each by itself or any combination thereof. Based on esophageal biopsies and endoscopic markers of severity, this panel of markers predicts the severity of fibrosis associated with esophagitis. Early detection of fibrosis with appropriate therapy may alter the natural course of the fibrotic process and prevent the development of severe scarring/stricture formation or even malignancy. This panel of markers is not limited to the esophagus but can include esophageal washings, and samples including serum/plasma, stool, and/or urine and/or other levels of the gastrointestinal tract.

Patients who undergo esophageal biopsy will have superficial esophageal epithelial tissue available for analysis and can have blood or other less invasive biomarkers assessed. This panel of markers will be used to predict the presence and/or severity of remodeling and fibrosis of the esophagus using an assessment of epithelial tissue from biopsies or other biomarkers found in less invasively obtained samples such as blood. There is currently no existing art for predicting the severity of fibrosis or dysfunction in the esophagus.

The disclosure shows that immunostaining of the esophagus biological sample with a panel of commercially available antibodies in a unique combination correlates with the degree of fibrosis using a specific scoring system. These antibodies have not previously been used to identify underlying fibrosis in EoE. The disclosure provides antibodies which are shown either in isolation or in combination to correlate with the degree of organ fibrosis. The epithelial staining studies using these antibodies have yielded data and correlate quantitatively with the degree of underlying fibrosis. The staining can be assessed to detect the amount of staining relative to the epithelial height/tissue. A threshold of color is set and only the color above the threshold is used to quantify the total stain.

Accordingly, the disclosure includes methods of diagnosing eosinophilic diseases and disorders such as EoE in a subject, wherein the methods comprise obtaining a sample from the subject and determining the presence and/or level of a diagnostic panel that contains markers selected from COMP-1 and one or more of tryptase and plasminogen activator inhibitor-1.

The disclosure also provide methods and determining the level of fibrosis or degree of an eosinophilic disease or disorder using one or more of the biomarkers selected from COMP-1, tryptase and plasminogen activator inhibitor-1 (PAI-1). The method includes measuring a diagnostic panel that contains the markers or genes selected from COMP-1, tryptase and PAI-1, analyzing the results to determine expression levels of the markers, and making a determination as to the eosinophilic disease or disorder (e.g., EoE) status of the subject based upon the expression levels of the markers. For example, Epithelial PAI-1 was significantly higher in active versus inactive EoE (p<0.001) and discriminated between fibrosis scores of 0 or 1 versus 3 and fibrosis score of 1 versus 2 (p<0.05 for each). Tryptase was significantly higher in active versus inactive EoE (p<0.0001) and differentiated between fibrosis score of 0 versus 1, 2, or 3 and a score of 1 versus 3 (p<0.05 for each). PAI-1 and tryptase correlated with fibrosis scores (r=0.62 and 0.76, respectively, p<0.0001). In another example, COMP expression was significantly induced (up to 6000 fold, p<0.0001) in EoE fibroblasts and esophageal epithelial cells treated with TGFb1. Staining of biopsy specimens (n=52) demonstrated elevated levels of COMP in the epithelium of active EoE patients as compared with inactive or normal patient biopsies (p<0.05 and <0.1, respectively). COMP expression correlated with fibrosis (r=0.63, p<001), basal zone hyperplasia (r=0.59, p<0.0001), and eosinophilia (r=0.54, p<0.0001). Other markers that can be used are provided in Table 1 (below).

For example, the methods of the disclosure can be used to identify active vs. inactive eosinophilic disease and disorders by measuring the percent staining of the sample. Generally, PAI-1 in Inactive EoE has a percent stain of: 9-13%; while active EoE has a percent stain of: 16% or more (e.g., 16-24%). When the staining is correlated with proximal compliance of the esophagus, inactive EoE showed a percent stain of: 8-19%; while active EoE has a percent stain of: 14% or greater (e.g., 14-28% or more). When the staining is correlated with mid-esophagus compliance in EoE subjects, where normal compliance is >2.6, and disease compliance is <2.6, inactive EoE has a percent stain of 6-17%, while active EoE has a percent stain of 14% or greater (e.g., about 14-27% or greater). When the staining is correlated with mid-esophagus compliance in all subjects (i.e., EoE and control), where normal compliance is >2.6, and disease compliance is <2.6, inactive EoE has a percent stain of 5-12%, while active EoE has a percent stain of 13% or more (e.g., 13-26% or greater).

The methods of the disclosure can also be used to distinguish eosinophilic disease and disorders from other inflammatory disorders in a subject, wherein the methods comprise measuring the expression profile of markers selected from the group consisting of COMP-1, tryptase, PAI-1 and any combination thereof, analyzing the results to determine expression levels of the markers, and making a determination as to the disease or disorder based upon the expression levels of the markers.

The disclosure also includes methods of determining the prognosis or treatment of a subject with an eosinophilic disease or disorder comprising measuring a panel of biomarkers selected from the group consisting of COMP-1, tryptase, PAI-1 and any combination thereof before and after a treatment regimen, wherein a beneficial change is indicative that the therapy is working to treat the disease or disorder when the presence of any of the markers decreases compared to an earlier measurement. In one embodiment, a beneficial change can be determined by a reduction in PAI-1 and/or tryptase expression or levels.

The disclosure also provides kits for the detection of a level of one or more markers associated with an eosinophilic disease or disorder (e.g., EoE) selected from the group consisting of COMP-1, tryptase, PAI-1 and any combination thereof. The kit can comprise one or more oligonucleotide probes complementary to subsequences of said one or more markers or genes. In some embodiments, the one or more probes are used in at least one of a gene chip, an expression array-based protocol, a PCR protocol, or an RNA level-based protocol, including, for example, RNA-seq, and the like. In another embodiment, the kit comprises antibodies and detection reagents for determining and antibody complex formed by the reagents.

In some embodiments, the markers or genes are measured using a microfluidic device. In other embodiments, the markers can be quantitatively measured using various systems known in the art, including, but not limited to, Taqman (Life Technologies, Carlsbad, Calif.), Light-Cycler (Roche Applied Science, Penzberg, Germany), ABI fluidic card (Life Technologies), NANOSTRINGS (NanoString Technologies, Seattle, Wash.), NANODROP technology (Thermo Fisher Scientific (Wilmington, Del.), Illumina technologies and the like. The person of skill in the art will recognize such other formats and tools, which can be commercially available or which can be developed specifically for such analysis.

In some embodiments of the disclosure, the diagnostic panel contains at least one marker selected from the group consisting of COMP-1, tryptase, PAI-1 and any combination thereof and can include additional markers associated with inflammation and the like. For example, the panel can include 1, 2, 3, 4, 5, 6, 7, 8, or 9 markers. In some embodiments the diagnostic panel contains 10, 11, 12, 13, 14, 15, 16, 17, 18, or 19 markers or genes. In some embodiments, the diagnostic panel contains 20, 21, 22, 23, 24, 25, 26, 27, 28, or 29 markers. In some embodiments, the diagnostic panel contains 30 to 50, 50 to 100, 100 to 200, 200 to 400 or 400 to 1000 markers or genes.

One embodiment of what is disclosed is the measurement of at least one or a panel of biomarkers with the selectivity and sensitivity required for managing and diagnosing subjects that have or may have a predisposition to a eosinophilic disease or disorder such as EoE.

As mentioned above, a probe or reagent used in detecting a marker typically comprises a molecule which can detectably distinguish changes in gene expression or can distinguish between target molecules differing in structure. Detection can be accomplished in a variety of different ways depending on the type of probe used and the type of target molecule. Examples of such specific binding include antibody binding and nucleic acid probe hybridization. Thus, for example, probes can include enzyme substrates, antibodies and antibody fragments, and nucleic acid hybridization probes (including primers useful for polynucleotide amplification and/or detection). Thus, in one embodiment, the detection of the presence or absence or amount of the at least one target polynucleotide biomarker involves contacting a biological sample with a probe, typically an oligonucleotide probe, where the probe hybridizes with the target polynucleotide in the biological sample containing a complementary sequence, where the hybridization is carried out under selective hybridization conditions. Such an oligonucleotide probe can include one or more nucleic acid analogs, labels or other substituents or moieties so long as the base-pairing function is retained.

Methods known in the art can be used to quantitatively measure the amount of mRNA transcribed by cells present in a sample. Examples of such methods include quantitative polymerase chain reaction (PCR), northern and southern blots, next generation sequencing. PCR allows for the detection and measurement of very low quantities of mRNA using an amplification process. Genes may either be up regulated or down regulated in any particular biological state, and hence mRNA levels shift accordingly.

In one embodiment, a method for gene expression profiling comprises measuring mRNA levels for biomarkers selected in a panel. Such a method can include the use of primers, probes, enzymes, and other reagents for the preparation, detection, and quantitation of mRNA (e.g., by PCR, by Northern blot and the like). In addition to the primers, reagents such as a dinucleotide triphosphate mixture having all four dinucleotide triphosphates (e.g., dATP, dGTP, dCTP, and dTTP), a reverse transcriptase enzyme, and a thermostable DNA polymerase were used for RT-PCR. Additionally buffers, inhibitors and activators can also be used for the RT-PCR process. Once the cDNA has been sufficiently amplified to a specified end point, the cDNA sample can be prepared for detection and quantitation. Though a number of detection schemes are contemplated, as will be discussed in more detail below, one method contemplated for detection of polynucleotides is fluorescence spectroscopy, and therefore labels suited to fluorescence spectroscopy are desirable for labeling polynucleotides. One example of such a fluorescent label is SYBR Green, though numerous related fluorescent molecules are known including, without limitation, DAPI, Cy3, Cy3.5, Cy5, CyS.5, Cy7, umbelliferone, fluorescein, fluorescein isothiocyanate (FITC), rhodamine, dichlorotriazinylamine fluorescein, dansyl chloride or phycoerythrin.

In another embodiment, a substrate comprising a plurality of oligonucleotide primers or probes may be used either for detecting or amplifying targeted sequences. The oligonucleotide probes and primers can be attached in contiguous regions or at random locations on the solid support. Alternatively the oligonucleotides may be attached in an ordered array wherein each oligonucleotide is attached to a distinct region of the solid support which does not overlap with the attachment site of any other oligonucleotide. Typically, such oligonucleotide arrays are “addressable” such that distinct locations are recorded and can be accessed as part of an assay procedure. The knowledge of the location of oligonucleotides on an array make “addressable” arrays useful in hybridization assays. For example, the oligonucleotide probes can be used in an oligonucleotide chip such as those marketed by Affymetrix and described in U.S. Pat. No. 5,143,854; PCT publications WO 90/15070 and 92/10092, the disclosures of which are incorporated herein by reference. These arrays can be produced using mechanical synthesis methods or light directed synthesis methods which incorporate a combination of photolithographic methods and solid phase oligonucleotide synthesis.

The immobilization of arrays of oligonucleotides on solid supports has been rendered possible by the development of a technology generally referred to as “Very Large Scale Immobilized Polymer Synthesis” in which probes are immobilized in a high density array on a solid surface of a chip (see, e.g., U.S. Pat. Nos. 5,143,854; and 5,412,087 and in PCT Publications WO 90/15070, WO 92/10092 and WO 95/11995, each of which are incorporated herein by reference), which describe methods for forming oligonucleotide arrays through techniques such as light-directed synthesis techniques.

In another embodiment, an array of oligonucleotides complementary to subsequences of the target gene are used to determine the identity of the target, measure its amount, and detect differences between the target and a reference wild-type sequence.

Hybridization techniques can also be used to identify the biomarkers of the disclosure and thereby determine or predict an eosinophilic disease or disorder. In this aspect, expression profiles or polymorphism(s) are identified based upon the higher thermal stability of a perfectly matched probe compared to the mismatched probe. The hybridization reactions may be carried out in a solid support (e.g., membrane or chip) format, in which, for example, the target nucleic acids are immobilized on nitrocellulose or nylon membranes and probed with oligonucleotide probes of the disclosure. Any of the known hybridization formats may be used, including Southern blots, slot blots, “reverse” dot blots, solution hybridization, solid support based sandwich hybridization, bead-based, silicon chip-based and microtiter well-based hybridization formats.

Hybridization of an oligonucleotide probe to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Oligonucleotides may be synthesized directly on the solid support or attached to the solid support subsequent to synthesis. Solid-supports suitable for use in detection methods of the disclosure include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

In one embodiment, a sandwich hybridization assay comprises separating the variant and/or wild-type target nucleic acid biomarker in a sample using a common capture oligonucleotide immobilized on a solid support and then contact with specific probes useful for detecting the variant and wild-type nucleic acids. The oligonucleotide probes are typically tagged with a detectable label.

Hybridization assays based on oligonucleotide arrays rely on the differences in hybridization stability of short oligonucleotides to perfectly matched and mismatched target variants. Each DNA chip can contain thousands to millions of individual synthetic DNA probes arranged in a grid-like pattern and miniaturized to the size of a dime or smaller. Such a chip may comprise oligonucleotides representative of both a wild-type and variant sequences.

Oligonucleotides of the disclosure can be designed to specifically hybridize to a target region of a polynucleotide. As used herein, specific hybridization means the oligonucleotide forms an anti-parallel double-stranded structure with the target region under certain hybridizing conditions, while failing to form such a structure when incubated with a different target polynucleotide or another region in the polynucleotide or with a polynucleotide lacking the desired locus under the same hybridizing conditions. Typically, the oligonucleotide specifically hybridizes to the target region under conventional high stringency conditions.

A nucleic acid molecule such as an oligonucleotide or polynucleotide is said to be a “perfect” or “complete” complement of another nucleic acid molecule if every nucleotide of one of the molecules is complementary to the nucleotide at the corresponding position of the other molecule. A nucleic acid molecule is “substantially complementary” to another molecule if it hybridizes to that molecule with sufficient stability to remain in a duplex form under conventional low-stringency conditions. Conventional hybridization conditions are described, for example, in Sambrook et al., Molecular Cloning, A Laboratory Manual, 2nd ed., Cold Spring Harbor Press, Cold Spring Harbor, N.Y. (1989), and in Haymes et al., Nucleic Acid Hybridization, A Practical Approach, IRL Press, Washington, D.C. (1985). While perfectly complementary oligonucleotides are used in most assays for detecting target polynucleotides or polymorphisms, departures from complete complementarity are contemplated where such departures do not prevent the molecule from specifically hybridizing to the target region. For example, an oligonucleotide primer may have a non-complementary fragment at its 5′ or 3′ end, with the remainder of the primer being complementary to the target region. Those of skill in the art are familiar with parameters that affect hybridization; such as temperature, probe or primer length and composition, buffer composition and salt concentration and can readily adjust these parameters to achieve specific hybridization of a nucleic acid to a target sequence.

A variety of hybridization conditions may be used in the disclosure, including high, moderate and low stringency conditions; see for example Maniatis et al., Molecular Cloning: A Laboratory Manual, 2d Edition, 1989, and Short Protocols in Molecular Biology, ed. Ausubel, et al., hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength and pH. The Tₘ is the temperature (under defined ionic strength, pH and nucleic acid concentration) at which 50% of the probes complementary to the target hybridize to the polyadenylated mRNA target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of helix destabilizing agents such as formamide. The hybridization conditions may also vary when a non-ionic backbone, i.e., PNA is used, as is known in the art. In addition, cross-linking agents may be added after target binding to cross-link, i.e., covalently attach, the two strands of the hybridization complex.

In another embodiment, a method for protein expression profiling comprises using one or more (e.g., a plurality of) antibodies to one or more biomarkers (e.g., COMP-1, tryptase, PAI-1 and any combination thereof) for measuring targeted polypeptide levels from a biological sample. In one embodiment contemplated for the method, the antibodies for the panel are bound to a solid support. The method for protein expression profiling may use a second antibody having specificity to some portion of the bound polypeptide. Such a second antibody may be detectably labeled with molecules useful for detection and quantitation of the bound polypeptides. Additionally, other reagents are contemplated for detection and quantitation including, for example, small molecules such as cofactors, substrates, complexing agents, and the like, or large molecules, such as lectins, peptides, olionucleotides, and the like. Such moieties may be either naturally occurring or synthetic.

The disclosure also contemplates the use of immunoassay techniques for measurement of polypeptide biomarkers identified herein. The polypeptide biomarker can be isolated and used to prepare antisera and monoclonal antibodies that specifically detect a biomarker gene product. Mutated gene products also can be used to immunize animals for the production of polyclonal antibodies. Recombinantly produced peptides can also be used to generate antibodies. For example, a recombinantly produced fragment of a polypeptide can be injected into a mouse along with an adjuvant so as to generate an immune response. Murine immunoglobulins which bind the recombinant fragment with a binding affinity of at least 1×10⁷ M⁻¹ can be harvested from the immunized mouse as an antiserum, and may be further purified by affinity chromatography or other means. Additionally, spleen cells are harvested from the mouse and fused to myeloma cells to produce a bank of antibody-secreting hybridoma cells. The bank of hybridomas can be screened for clones that secrete immunoglobulins which bind the recombinantly produced fragment with an affinity of at least 1×10⁶ M⁻¹. More specifically, immunoglobulins that selectively bind to the variant polypeptides but poorly or not at all to wild-type polypeptides are selected, either by pre-absorption with wild-type proteins or by screening of hybridoma cell lines for specific idiotypes that bind the variant, but not wild-type, polypeptides.

Biomarkers, including proteins or nucleic acids, can be detected or measured using methods generally known in the art. Expression levels/amount of a gene or a biomarker can be determined based on any suitable criterion known in the art, including but not limited to mRNA, cDNA, proteins, protein fragments and/or gene copy number. Methods of detection generally encompass methods to quantify the level of a biomarker in the sample (quantitative method) or that determine whether or not a biomarker is present in the sample (qualitative method). It is generally known to the skilled artisan which of the following methods are suitable for qualitative and/or for quantitative detection of a biomarker. Samples can be conveniently assayed for, e.g., proteins using Westerns and immunoassays, like ELISAs, RIAs, fluorescence-based immunoassays, as well as mRNAs or DNAs from a genetic biomarker of interest using Northern, dot-blot, polymerase chain reaction (PCR) analysis, array hybridization, RNase protection assay, or using DNA SNP chip microarrays, which are commercially available, including DNA microarray snapshots. Further suitable methods to detect biomarker include measuring a physical or chemical property specific for the peptide or polypeptide such as its precise molecular mass or NMR spectrum. Said methods comprise, e.g., biosensors, optical devices coupled to immunoassays, biochips, analytical devices such as mass-spectrometers, NMR-analyzers, or chromatography devices. Further, methods include microplate ELISA-based methods, fully-automated or robotic immunoassays, CBA (an enzymatic Cobalt Binding Assay), and latex agglutination assays.

For the detection of biomarker proteins a wide range of immunoassay techniques using such an assay format are available, see, e.g., U.S. Pat. Nos. 4,016,043, 4,424,279, and 4,018,653. These include both single-site and two-site or “sandwich” assays of the non-competitive types, as well as in the traditional competitive binding assays. These assays also include direct binding of a labeled antibody to a target biomarker. In certain embodiments, the expression of proteins in a sample is examined using immunohistochemistry (“IHC”) and staining protocols. Immunohistochemical staining of tissue sections has been shown to be a reliable method of assessing or detecting presence of proteins in a sample. Immunohistochemistry techniques utilize an antibody to probe and visualize cellular antigens in situ, generally by chromogenic or fluorescent methods.

In a specific embodiment, the detection of a marker (e.g., PAI-1, COMP-1, tryptase etc.) comprises a histochemical staining procedure (such as immunohistochemistry or an analogous procedure using other entities specific for the protein biomarker). In certain embodiments, the biomarker specific reagents are deposited on a sample to be analyzed. In a specific embodiment, the biomarker-specific agent is an antibody, and the biomarker-specific antibodies are deposited on a sample. The sample can be a formalin fixed and/or paraffin-embedded sample.

Biomarker-specific reagents are visualized using detection reagents to deposit a detectable entity that generates a detectable signal associated with the biomarker. When associated with a biomarker-specific reagent (either directly or indirectly), the detectable signal can be used to locate and/or quantify the biomarker to which the biomarker-specific reagent is directed. Thereby, the presence and/or concentration of the target in a sample can be detected by detecting the signal produced by the detectable entity. A detectable signal can be generated by any mechanism including absorption, emission and/or scattering of a photon (including radio frequency, microwave frequency, infrared frequency, visible frequency and ultra-violet frequency photons). Detectable entities include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected through antibody-hapten binding interactions using additional detectably labeled antibody conjugates, and paramagnetic and magnetic molecules or materials. Particular examples of detectable entities include enzymes such as horseradish peroxidase, alkaline phosphatase, acid phosphatase, glucose oxidase, (3-galactosidase or 3-glucuronidase; fluorphores such as fluoresceins, luminophores, coumarins, BODIPY dyes, resorufins, and rhodamines (many additional examples of fluorescent molecules can be found in The Handbook—A Guide to Fluorescent Probes and Labeling Technologies, Molecular Probes, Eugene, Oreg.); nanoparticles such as quantum dots (obtained, for example, from QuantumDot Corp, Invitrogen Nanocrystal Technologies, Hayward, Calif.; see also, U.S. Pat. Nos. 6,815,064, 6,682,596 and 6,649,138, each of which patents is incorporated by reference herein); metal chelates such as DOTA and DPTA chelates of radioactive or paramagnetic metal ions like Gd 3+; and liposomes, for example, liposomes containing trapped fluorescent molecules. Where the detectable entity includes an enzyme, a detectable substrate such as a chromogen, a fluorogenic compound, or a luminogenic compound can be used in combination with the enzyme to generate a detectable signal. Particular examples of chromogenic compounds include diaminobenzidine (DAB), 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2T-azino-di-[3-ethylbenzothiazoline sulphonate] (ABTS), o-dianisidine, 4-chloronaphthol (4-CN), nitrophenyl-β-D-galactopyranoside (ONPG), o-phenylenediamine (OPD), 5-bromo-4-chloro-3-indolyl-.beta.-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue and tetrazolium violet. Alternatively, an enzyme can be used in a metallographic detection scheme. Metallographic detection methods include using an enzyme such as alkaline phosphatase in combination with a water-soluble metal ion and a redox-inactive substrate of the enzyme. The substrate is converted to a redox-active agent by the enzyme, and the redox-active agent reduces the metal ion, causing it to form a detectable precipitate. Metallographic detection methods include using an oxido-reductase enzyme (such as horseradish peroxidase) along with a water soluble metal ion, an oxidizing agent and a reducing agent, again to form a detectable precipitate. Haptens are small molecules that are specifically bound by antibodies, although by themselves they will not elicit an immune response in an animal and must first be attached to a larger carrier molecule such as a protein to generate an immune response. Examples of haptens include di-nitrophenyl, biotin, digoxigenin, and fluorescein.

In a specific embodiment, the biomarker-specific reagent is an antibody (termed “primary antibody”) and the detection reagents include an antibody capable of binding to the primary antibody (termed “secondary antibody”) and a detectable entity including an enzyme coupled to or adapted to be coupled to the secondary antibody and reagents reactive with the enzyme to deposit a chromogen or fluorophore on the sample. In an embodiment, the secondary antibody has affinity for immunoglobulins from a specific animal species from which the primary antibody is derived (termed a “species-specific secondary antibody”). In another embodiment, the secondary antibody is reactive with a tag incorporated into the primary antibody, such as an epitope tag located in the primary amino acid sequence of the primary antibody or a hapten coupled to a reactive side chain of the primary antibody.

The level of staining can be scored both for staining intensity and percent staining through the use of automated systems and optionally confirmed by a pathologist.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, -galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

Moreover, detectable labels may be measured using CCD cameras that convert an optical level to a digital signature.

It is also contemplated that the gene expression profile or antibody detection methods may be transmitted to a remote location for analysis. For example, changes in a detectable signal related to gene expression or protein levels from a first time and a second time are communicated to a remote location for analysis by a technician or clinician.

The digital representation of the detectable signal is transmittable over any number of media. For example, such digital data can be transmitted over the Internet in encrypted or in publicly available form. The data can be transmitted over phone lines, fiber optic cables or various air-wave frequencies. The data are then analyzed by a central processing unit at a remote site, and/or archived for compilation of a data set that could be mined to determine, for example, changes with respect to historical mean “normal” values of a genetic expression profile of a subject.

Embodiments of the disclosure include systems (e.g., internet based systems), particularly computer systems which store and manipulate the data corresponding to the detectable signal obtained an expression profile. As used herein, “a computer system” refers to the hardware components, software components, and data storage components used to analyze the digital representative of an expression profile or plurality of profiles. The computer system typically includes a processor for processing, accessing and manipulating the data. The processor can be any well-known type of central processing unit.

Typically the computer system is a general purpose system that comprises the processor and one or more internal data storage components for storing data, and one or more data retrieving devices for retrieving the data stored on the data storage components. A skilled artisan can readily appreciate that any one of the currently available computer systems are suitable.

In one particular embodiment, the computer system includes a processor connected to a bus which is connected to a main memory (preferably implemented as RAM) and one or more internal data storage devices, such as a hard drive and/or other computer readable media having data recorded thereon. In some embodiments, the computer system further includes one or more data retrieving device for reading the data stored on the internal data storage devices.

The data retrieving device may represent, for example, a floppy disk drive, a compact disk drive, a magnetic tape drive, or a modem capable of connection to a remote data storage system (e.g., via the internet) and the like. In some embodiments, the internal data storage device is a removable computer readable medium such as a floppy disk, a compact disk, a magnetic tape, and the like, containing control logic and/or data recorded thereon. The computer system may advantageously include or be programmed by appropriate software for reading the control logic and/or the data from the data storage component once inserted in the data retrieving device.

### EXAMPLES

### Example 1

Eosinophilic esophagitis (EoE) pathogenesis includes subepithelial fibrosis of the lamina propria (LP). Fibrosis can be scored semi-quantitatively with a fibrosis score. While fibrosis is thought to reflect disease severity, not all biopsies contain adequate LP for analysis. We have previously shown that PAI-1 is part of the EoE fibrotic cascade. Immunohistological patterns of epithelial staining for plasminogen activator inhibitor-1 (PAI-1) and tryptase in the esophageal epithelium were investigated as potential markers of fibrotic severity.

Esophageal tissue biopsies from children with active and inactive EoE and from normal esophagi were stained with PAI-1 and tryptase (n=41 PAI-1, n=57 tryptase). The degree of active staining was quantified in the epithelium using Image J. The relationship between PAI-1, tryptase, and fibrosis scores was evaluated.

Epithelial PAI-1 was significantly higher in active versus inactive EoE (p<0.001) and discriminated between fibrosis scores of 0 or 1 versus 3 and fibrosis score of 1 versus 2 (p<0.05 for each). Tryptase was significantly higher in active versus inactive EoE (p<0.0001) and differentiated between fibrosis score of 0 versus 1, 2, or 3 and a score of 1 versus 3 (p<0.05 for each). PAI-1 and tryptase correlated with fibrosis scores (r=0.62 and 0.76, respectively, p<0.0001).

Epithelial staining patterns for PAI-1 and tryptase distinguish distinct groups of fibrotic severity and poses a way to gauge the potential severity of EoE in a patient through immunohistochemistry of the epithelium.

### Example 2

Esophageal remodeling in eosinophilic esophagitis (EoE) causes progressive luminal narrowing involving subepithelial fibrosis with smooth muscle hypertrophy and dysfunction. The molecular mechanisms of remodeling continue to be elucidated. Cartilage oligomeric matrix protein (COMP) is involved tissue fibrosis in diseases such as pulmonary fibrosis.

Primary esophageal fibroblasts from EoE patients were used. The fibroblast were treated in vitro with recombinant TGFb1. Biopsies from children with active and inactive EoE as well as normal control biopsies were stained for COMP, expression was quantified using Image J, and compared with histology scores. Biopsies were scored using a standardized histology score.

COMP expression was significantly induced (up to 6000 fold, p<0.0001) in EoE fibroblasts and esophageal epithelial cells treated with TGFb1. Staining of biopsy specimens (n=52) demonstrated elevated levels of COMP in the epithelium of active EoE patients as compared with inactive or normal patient biopsies (p<0.05 and <0.1, respectively). COMP expression correlated with fibrosis (r=0.63, p<001), basal zone hyperplasia (r=0.59, p<0.0001), and eosinophilia (r=0.54, p<0.0001).

COMP is induced in EoE fibroblasts by TGFb1 and elevated in EoE biopsies from children with active disease. Since it correlates with basal zone hyperplasia and fibrosis scores, COMP may function as a marker of EoE severity.

The canonical pro-fibrotic molecule, transforming growth factor-beta-1 (TGFβ1) induces epithelial and fibroblast expression of markers in the EoE esophagus that correlate with the degree of fibrosis in the subepithelial lamina propria. A combination of these markers may function to predict fibrotic severity. Two of the markers in the proposed panel (serpinE1/plasminogen activator inhibitor-1 (PAI-1)) and COMP are induced by TGFβ1. The third proposed marker, tryptase, is derived from mast cells. COMP correlates with fibrosis with a Spearman's coefficient “r” of 0.63 (p<0.0001). PAI-1 and tryptase correlate positively with fibrosis to similar degrees as COMP with Spearman's “r” of 0.62 and 0.76 (p<0.0001), respectively (Aceves, Dohil, unpublished). Using the 3 markers together (or in some other combination with or without other candidate markers) will increase the sensitivity of reflecting fibrosis. Epithelial PAI-1 discriminates between the standardized fibrosis scores of 0 or 1 from a score of 3. It also discriminates between a fibrosis score of 1 versus 2. In contrast tryptase differentiates between fibrosis score of 0 versus 1, 2, or 3. COMP distinguished fibrosis scores of 0 or 1 versus 3. As such, each of these epithelial stains has a distinct range of distinguishing fibrosis scores in the esophagus.

Based on recent discovery datasets (Table 1) using EoE and normal fibroblasts, further experiments are planned for a set of markers to understand if they are expressed differentially in EoE as compared to normal tissues, their pattern of tissue expression in esophageal biopsies, and the ability to find them in plasma samples. Each marker will be analyzed for its esophageal and plasma expression levels and assessed for its relationship with fibrosis score, esophageal eosinophilia, and other markers of histological disease. A panel of these markers in the esophagus and/or plasma will correlate with the degree of esophageal fibrosis and other histological severity markers as well as endoscopic parameters of disease severity and/or tissue remodeling. Additional discovery datasets derived from RNA sequencing are currently under analysis and will serve as hypothesis generating for future potential surrogate fibrotic and/or disease severity markers.

TABLE 1
Proteomic Analysis of EoE and Normal Fibroblasts

| | RATIO EoE TO |
|---|---|
| GENE | NORMAL |
| TGM2 | 2.11 |
| NEST | 2.27 |
| TSP1 | 1.63 |
| NQO1 | 1.87 |
| CD44 | 1.72 |
| CD166 | 1.68 |
| TF | 1.84 |
| CD14 | 1.9 |
| MGLL | 1.79 |
| RS26 | 1.66 |
| S10A4 | 2.67 |
| COL6 | 1.19 |

### Example 3

Functional Lumen Imaging Probe (EndoFlip) technology has recently been used to evaluate compliance and distensibility of the human esophagus in a number of diseases including EoE. EndoFlip can be used in children with EoE to quantify the degree of esophageal fibrotic and epithelial remodeling and in particular that compliance is a more sensitive measure of underlying remodeling than is distensibilty. The EndoFlip is able to measure compliance in the proximal, mid and distal esophagus.

In children, strictures begin in the proximal esophagus. The EndoFLIP studies demonstrate that proximal compliance best distinguishes EoE from non-EoE patients in children. Using a cohort of normal and EoE children, a cut-off of 2.6 can distinguish normal from diseased esophagi. In order to understand which histologic markers best align with proximal compliance, COMP, PAI-1, and tryptase were evaluate individually and in combinations. Isolated mid esophageal PAI-1 staining best distinguished active from inactive EoE (p=0.02). Mid esophageal COMP and tryptase distinguished active from inactive EoE (p=0.04 for both). PAI-1 staining in the mid esophagus was able to distinguish both overall and proximal compliance.

The range of PAI-1 staining quantified using the computer program ImageJ demonstrated levels of 0.01-0.13 (25, 75th percentiles) in Inactive EoE (EoE-IA) and 0.16-0.24 in Active EoE (EoE-A). These data show that PAI-1 distinguishes active and inactive EoE.

Unlike PAI-1, compliance was not able to distinguish EoE-IA from EoE-A suggesting that compliance would not be an adequate isolated gauge of EoE state. PAI-1 staining was able to not only distinguish EoE-A from EoE-IA but also EoE pateints with and without normal compliance (p=0.04). Given these findings PAI-1 staining at a single level of the esophagus has the capacity to estimate not only inflammatory disease activity but also esophageal remodeling reflected in the EndoFLIP. Lastly, PAI-1 demonstrated a strong correlation with subepithelial fibrosis (r=0.68, p=0.0005). By extension, then, a single mid esophageal biopsy stained for PAI-1 can function as an indicator of both active EoE and esophageal compliance.

Given these data that PAI-1 distinguishes 1) active from inactive EoE, 2) normal from abnormal esophageal compliance, and 3) correlates with fibrosis, the data demonstrate that PAI-1 can be used as a marker to 1) decide on a therapeutic algorithm for EoE treatment such that more severe PAI-1 staining would justify the need for more aggressive interventions such as combination therapy and 2) to gauge the success of therapies on the EoE complication of esophageal remodeling. This has immediate applicability due to the current lack of markers of EoE severity.

TABLE 2
Aggregate regression coefficients, Perason
correlations, and Spearman correlations:

| | β | Pearson | Spearman |
|---|---|---|---|
| Overall Compliance | | | |
| Avg. PAI | −5.02 | −0.28; 0.031* | −0.35; 0.007* |
| Prox. PAI | −1.82 | −0.14; 0.304 | −0.07; 0.611 |
| Prox. Mid. PAI | −3.72 | −0.24; 0.076 | −0.27; 0.046* |
| Mid. PAI | −4.09 | −0.28; 0.037* | −0.33; 0.012* |
| Distal PAI | −4.95 | −0.30; 0.023* | −0.29; 0.025* |
| Avg. COMP | −2.06 | −0.11; 0.5 [symbol] 9 | 0.07; 0.707 |
| Prox. COMP | −0.43 | −0.02; 0.8 [symbol] 9 | 0.14; 0.405 |
| Mid. COMP | −2.32 | −0.26; 0.127 | −0.09; 0.596 |
| Distal COMP | −1.35 | −0.09; 0.588 | −0.02; 0.887 |
| Avg. Tryptase | −0.10 | −0.30; 0.086 | −0.35; 0.048* |
| Prox. Tryptase | −0.06 | −0.23; 0.080 | −0.30; 0.056 |
| Mid. Tryptase | −0.08 | −0.32; 0.053 | −0.46; 0.004** |
| Distal Tryptase | −0.05 | −0.20; 0.209 | −0.23; 0.134 |
| Prox. Mid. Compliance | | | |
| Avg. PAI | −6.60 | −0.33; 0.011* | −0.41; 0.001** |
| Prox. PAI | −2.44 | −0.16; 0.218 | −0.10; 0.465 |
| Prox. Mid. PAI | −4.85 | −0.27; 0.038* | −0.32; 0.016* |
| Mid. PAI | − [symbol] .35 | −0.32; 0.014* | −0.42; 0.001** |
| Distal PAI | − [symbol] .99 | −0.33; 0.011* | −0.36; 0.005** |
| Avg. COMP | −2.74 | −0.13; 0.479 | 0.09; 0.615 |
| Prox. COMP | −2.45 | −0.12; 0.48 [symbol] | 0.07; 0.673 |
| Mid. COMP | −2.27 | −0.23; 0.188 | −0.03; 0.854 |
| Distal COMP | −1.17 | −0.08; 0.640 | 0.01; 0.935 |
| Avg. Tryptase | −0.12 | −0.32; 0.071 | −0.32; 0.070 |
| Prox. Tryptase | −0.09 | −0.33; 0.038* | −0.29; 0.071 |
| Mid. Tryptase | −0.10 | −0.36; 0.031* | −0.46; 0.004** |
| Distal Tryptase | −0.04 | −0.17; 0.276 | −0.19; 0.224 |
| Proximal Compliance | | | |
| Avg. PAI | −8.50 | −0.36; 0.007** | −0.50; <0.001*** |
| Prox. PAI | −3.46 | −0.19; 0.159 | −0.23; 0.095 |
| Prox. Mid. PAI | −6.25 | −0.29; 0.031* | −0.47; <0.001*** |
| Mid. PAI | −6.65 | −0.33; 0.013* | −0.52; <0.001*** |
| Distal PAI | −7.3 [symbol] | −0.3 [symbol] ; 0.007** | −0.34; 0.010* |
| Avg. COMP | −4.42 | −0.16; 0.396 | 0.10; 0.575 |
| Prox. COMP | −6.56 | −0.24; 0.1 [symbol] 7 | −0.0 [symbol] ; 0.760 |
| Mid. COMP | −2.18 | −0.16; 0.3 [symbol] 8 | 0.02; 0.916 |
| Distal COMP | −2.51 | −0.13; 0.432 | −0.01; 0.965 |
| Avg. Tryptase | −0.15 | −0.28; 0.109 | −0.32; 0.073 |
| Prox. Tryptase | −0.10 | −0.28; 0.08 [symbol] | −0.29; 0.067 |
| Mid. Tryptase | −0.11 | −0.29; 0.0 [symbol] 3 | −0.42; 0.009** |
| Distal Tryptase | −0.07 | −0.22; 0.163 | −0.23; 0.139 |
| Mid. Compliance | | | |
| Avg. PAI | −5.27 | −0.23; 0.089 | −0.29; 0.027* |
| Prox. PAI | −1.83 | −0.10; 0.438 | 0.02; 0.902 |
| Prox. Mid. PAI | −4.11 | −0.20; 0.142 | −0.20; 0.145 |
| Mid. PAI | −4.45 | −0.23; 0.089 | −0.32; 0.014* |
| Distal PAI | −4.67 | −0.18; 0.166 | −0.21; 0.104 |
| Avg. COMP | −1.06 | −0.05; 0.800 | 0.12; 0.524 |
| Prox. COMP | 1.66 | 0.07; 0.662 | 0.23; 0.177 |
| Mid. COMP | −2.37 | −0.22; 0.199 | −0.08; 0.654 |
| Distal COMP | 0.18 | 0.01; 0.955 | 0.06; 0.707 |
| Avg. Tryptase | −0.10 | −0.24; 0.1 [symbol] 4 | −0.30; 0.090 |
| Prox. Tryptase | −0.07 | −0.2 [symbol] ; 0.095 | −0.23; 0.145 |
| Mid. Tryptase | −0.09 | −0.30; 0.075 | −0.40; 0.014* |
| Distal Tryptase | −0.02 | −0.05; 0.754 | −0.11; 0.496 |
| Distal Compliance | | | |
| Avg. PAI | −2.14 | −0.10; 0.478 | −0.10; 0.448 |
| Prox. PAI | −1.09 | −0.06; 0.630 | −0.01; 0.924 |
| Prox. Mid. PAI | −1.34 | −0.07; 0.619 | −0.08; 0.579 |
| Mid. PAI | −1.04 | −0.06; 0.683 | −0.10; 0.440 |
| Distal PAI | −4.80 | −0.14; 0.285 | −0.08; 0.570 |
| Avg. COMP | −2.68 | −0.12; 0507 | −0.02; 0.902 |
| Prox. COMP | 2.59 | 0.12; 0.495 | 0.22; 0.200 |
| Mid. COMP | −2.53 | −0.24; 0.174 | −0.11; 0.543 |
| Distal COMP | −4.26 | −0.13; 0.410 | −0.08; 0.620 |
| Avg. Tryptase | −0.14 | −0.35; 0.053 | −0.38; 0.032* |
| Prox. Tryptase | −0.04 | −0.15; 0.374 | −0.29; 0.074 |
| Mid. Tryptase | −0.06 | −0.20; 0.231 | −0.32; 0.052 |
| Distal Tryptase | −0.12 | −0.21; 0.179 | −0.27; 0.081 |

[symbol]

indicates data missing or illegible when filed

TABLE 3
Logistic regression output for modeling active vs. inactive subjects.

| | Estimate | Std. Error | z value | p-value | Odds Ratio | 95% Cl |
|---|---|---|---|---|---|---|
| Avg. PAI | | | | | | |
| (Intercept) | −9.182 | 3.123 | −2.940 | 0.003** | — | — |
| Avg. PAI | 6.393 | 2.135 | 2.994 | 0.003** | 597.708 | (22.449, 127963.127) |
| Prox. PAI | | | | | | |
| (Intercept) | −1.560 | 0.795 | −1.963 | 0.050* | — | — |
| Prox. PAI | 1.295 | 0.511 | 2.534 | 0.011* | 3.653 | (1.541, 11.650) |
| Prox. Mid. PAI | | | | | | |
| (Intercept) | −4.296 | 1.648 | −2.608 | 0.009** | — | — |
| Prox. Mid, PAI | 3.163 | 1.122 | 1.870 | 0.005** | 23.637 | (3.855, 337.,)08) |
| Mid. PAI | | | | | | |
| (Intercept) | −1.504 | 0.876 | −1.718 | 0.086 | — | — |
| Mid. PAI | 1.240 | 0.513 | 2.417 | 0.016* | 3.456 | (1.420, 10.933) |
| Distal PAI | | | | | | |
| (Intercept) | −2.289 | 0.840 | −2.725 | 0.006** | — | — |
| Distal PAI | 1.726 | 0.537 | 3.214 | 0.001** | 5.616 | (2.247, 19.126) |
| Avg. COMP | | | | | | |
| (Intercept) | −1.094 | 0.781 | −1.401 | 0.161 | — | — |
| Avg. COMP | 0.990 | 0.562 | 1.762 | 0.078 | 2.690 | (1.002, 9.457) |
| Prox. COMP | | | | | | |
| (intercept) | 0.294 | 0.537 | 0.548 | 0.584 | — | — |
| Prox. COMP | 0.011 | 0.416 | 0.027 | 0.979 | 1.011 | (0.445, 2.373) |
| Mid. COMP | | | | | | |
| (intercept) | -0.670 | 0.602 | −1.112 | 0.266 | — | — |
| Md. COMP | 0.658 | 0.330 | 1.991 | 0.046* | 1.930 | (1.121, 4.321) |
| Distal COMP | | | | | | |
| (intercept) | −0.499 | 0.576 | −0.865 | 0.387 | — | — |
| Distal COMP | 0.656 | 0.379 | 1.779 | 0.084 | 1.927 | (1.000, 4.542) |
| Avg. Tryptase | | | | | | |
| (Intercept) | −2.244 | 0.919 | −2.443 | 0.015* | — | — |
| Avg. Tryptase | 0.485 | 0.187 | 2.589 | 0.010** | 1.624 | (1.197, 2.534) |
| Prox. Tryptase | | | | | | |
| (Intercept) | −0.772 | 0.564 | −1.369 | 0.171 | — | — |
| Prox. Tryptase | 0.315 | 0.161 | 1.950 | 1.051 | 1.370 | (1.080, 2.023) |
| Mid. Tryptase | | | | | | |
| (Intercept) | −1.532 | 0.815 | −1.881 | 0.060 | — | — |
| Mid. Tryptase | 0.514 | 0.254 | 2.028 | 0.043* | 1.673 | (1.171, 3.090) |
| Distal Tryptase | | | | | | |
| (Intercept) | −0.518 | 0.512 | −1.012 | 0.311 | — | — |
| Distal Tryptase | 0.125 | 0.069 | 1.822 | 0.068 | 1.133 | (1.012, 1.333) |

What is claimed is:

1. A method comprising:
determining an eosinophilic disease or disorder status in a subject, comprising measuring the levels of a biomarker panel in a sample from the subject that comprises a plasminogen activator inhibitor 1 (PAI-1) polypeptide, marker or gene and optionally at least one polypeptide, marker or gene selected from the group consisting of cartilage oligomeric matrix protein-1 (COMP-1), tryptase and any combination thereof;
analyzing the result to determine a level of expression of the PAI-1 polypeptide, marker or gene;
determining the eosinophilic disease or disorder status of the subject based upon the level of the expression or marker, wherein a level of PAI-1 that is elevated compared to normal control is indicative of active eosinophilic disease or disorder; and
administering a therapeutic composition to a subject with active eosinophilic disease or disorder selected from oral viscous budesonide (OVB), steroids, OVB/rosiglitazone, systemic corticosteroids, reslizumab, dupilumab, anti-IL13 and any combination thereof.

2. The method of claim 1, wherein the determining of the eosinophilic disease or disorder status comprises determining the percent of epithelium that stained positively for PAI-1.

3. The method of claim 2, wherein a percent of staining for PAI-1 of greater than 14% is indicative of active eosinophilic disease or disorder.

4. The method of claim 1, wherein the eosinophilic disease or disorder status comprises a diagnosis of eosinophilic esophagitis.

5. The method of claim 1, wherein the at least one marker or gene comprises mRNA.

6. The method of claim 1, wherein the at least one marker or gene comprises protein.

7. The method of claim 1, wherein the subject is a human patient.

8. The method of claim 1, wherein the sample is obtained from the group consisting of a tissue, an exudate, saliva, serum, plasma, blood, oral, urine, stool, and a buccal sample.

9. The method of claim 8, wherein the sample is a tissue sample.

10. The method of claim 9, wherein the tissue sample is an esophageal tissue sample.

11. The method of claim 1, wherein the determining step comprises analyzing a subset of the markers or genes using at least one algorithm.

12. The method of claim 1, wherein the status comprises distinguishing eosinophilic esophagitis from a normal condition in the subject.

13. The method of claim 1, wherein the status comprises distinguishing eosinophilic esophagitis from at least one other eosinophilic disorder in the subject.

14. The method of claim 1, further comprising developing or modifying a therapy for the subject based upon the results of the diagnostic panel analysis.

15. The method of claim 1, wherein the sample is an archival sample.

16. The method of claim 15, wherein the archival sample is a formalin-fixed, paraffin-embedded (FFPE) sample.

17. The method of claim 1, further comprising determining and/or monitoring exposure to one or more therapeutic compounds in the subject based upon the level of expression.

18. A method comprising of identifying an active vs. inactive eosinophilic disease or disorder, comprising:
measuring the levels of a biomarker panel in a sample from the subject that comprises a plasminogen activator inhibitor 1 (PAI-1) polypeptide, marker or gene and optionally at least one polypeptide, marker or gene selected from the group consisting of cartilage oligomeric matrix protein-1 (COMP-1), tryptase and any combination thereof;
analyzing the result to determine a level of expression of the PAI-1 polypeptide, marker or gene and optionally COMP-1 and tryptase, wherein the level of expression is determine by an automated optical camera;
determining the eosinophilic disease or disorder status of the subject based upon the level of the expression or marker, wherein a level of PAI-1 and optionally COMP-1 and tryptase that is elevated compared to normal control is indicative of active eosinophilic disease or disorder.

19. The method of claim 18, wherein the PAI-1 level that is indicative of active eosinophilic disease or disorder comprises a stain percent of the sample of greater than 14%.

20. An EoE molecular diagnostic panel for use in the detection of at least one marker selected from COMP-1, tryptase and PAI-1.
